(19) United States
(12) Patent Application Publication
(10) Pub. No.: US 2024/0398504 A1
(43) Pub. Date: Dec. 5, 2024

(54) HOLDING SYSTEM FOR AN OPERATOR AND METHOD FOR HOLDING AN OPERATOR

(71) Applicant: HELLSTERN MEDICAL GMBH, Wannweil (DE)

(72) Inventors: Sabrina HELLSTERN, Wannweil (DE); Claudia SODHA, Gechingen (DE); Alexander STROBEL, Haigerloch-Stetten (DE); Harald RAGER, Bisingen-Wessingen (DE)

(21) Appl. No.: 18/837,727

(22) PCT Filed: Feb. 9, 2023
(86) PCT No.: PCT/EP2023/053179

§ 371 Date: Aug. 12, 2024

(30) Foreign Application Priority Data
Feb. 10, 2022 (DE) 10 2022 103 151.9

(51) Int. Cl. A61B 90/60

(52) U.S. Cl. CPC A61B 90/60

(57) ABSTRACT

A holding system, including a support device for holding at least one body part of the operator and a release device, functions to hold at least one body part of the operator in an adjustable position. The support device has a control unit which is designed to switch the support device between a moving mode and a holding mode, wherein the supported body part of the operator can be moved at least in an adjusting direction in the moving mode and is held in a position and/or orientation by the support unit in the holding mode. The control unit is designed to switch the support device from the moving mode into the holding mode if the supported body part of the operator remains in an at least substantially constant position and/or orientation for a predetermined time.

### CROSS REFERENCE TO RELATED APPLICATIONS

This application is a National Stage of PCT Application No. PCT/EP2023/053179 filed on Feb. 9, 2023, which claims priority to German Patent Application No. DE 10 2022 103 151.9 filed on Feb. 10, 2022, the contents each of which are incorporated herein by reference thereto.

### TECHNICAL FIELD

The present disclosure refers to a holding system for an operator in the surgery as well as, for example, also for laboratory personnel or dentists.

### BACKGROUND

Holding systems for operators, such as operating chairs with or without arm or foot rests for an operator, are already known and serve generally to support the operator normally standing at the operating table in a holding manner, so that he can carry out operations with long durations, operations in a forced posture and/or operations with special requirements to fine motor skills with reduced or without fatigue and can in the ideal case carry out the latter with reliable and high quality.

Operating chairs are usually adjustably configured in order to be adaptable to the needs of the operator during the operation. For example, an operating chair is known from DE 10 2020 103 861 B3 that can be adjusted in height as well as in a lateral direction and in forward-backward-direction in a pivoting manner.

DE 20 2018 102 964 U1 describes a height-adjustable operating chair having two foot pedals, wherein an adjustment of the height of the operating chair by the operator sitting on the operator chair can be carried out by means of the foot pedals. The operating chair can, in addition, store an adjusted height and be brought into the stored height when restarted.

Moreover, an operating chair having an arrestable hinge arm is disclosed in EP 0 868 885 A1, which is configured to support an arm of an operator sitting on the chair. The operator can adjust the inclination of the holding arm by means of a foot pedal. If the operator actuates the foot pedal, hydraulic oil is pumped by means of a hydraulic pump into a cylinder chamber, whereby a piston is tensioned against a spring element and a rotation of the hinge arm is released. As soon as the operator takes his foot from the foot pedal, the hydraulic pump stops, so that the spring element pushes the piston back and blocks the hinge arm.

Moreover, an operating chair arranged on a drivable platform for use in laparoscopic operations is described in US 2016/0000631. The operating chair comprises a seat supported by a column, in which a chest support towering at the front of the seat comprises multiple adjustable support pads. The chest support can be adjusted, so that a most ergonomic seating position shall be achieved for the operator. For the adjustment of the sitting position, it is thereby necessary to make inputs into the system prior to the operation relating to the position of the patient to be operated, to biometric information about the operator and to the desired position of the operator, such as the seat height, the seat inclination, or the desired height of the foot rest above the platform. Based on this information, the operating chair is then calibrated.

During the operation it can happen that the operator has to change his position relative to the patient frequently, so that the operating chair has to be readjusted accordingly.

Considering an increasing number of functions and adjustment possibilities of operating chairs, a manual adjustment is obvious, also an adjustment by means of an operating device that can be manually operated by the operator, because an adjustment by foot can result in an incorrect adjustment of the operating chair in case of a high number of operating elements.

Adjusting the operating chair manually results, however, in that the operator has to lay down the operating instrument held in his hand, and thus has to interrupt the operation process. Only after the adjustment of the operating chair, the operator can continue with the operation. The operator is thus torn out of the operation process due to the manual adjustment of the operating chair. In operations in which a multiplicity of such position changes of the operator is necessary, an elongation of the operation can result.

Starting therefrom, it is the object of the present disclosure to provide a system and a method that allow to hold the body of the operator in a multiplicity of different positions that are adjustable as simple and intuitively adjustable as possible.

This object is solved by means of a holding system for an operator including: a support device for holding at least one body part of the operator, which comprises a control device that is configured to switch the support device between a movement mode and a holding mode, wherein the supported body part of the operator can be moved in an adjustment direction in the movement mode and is held in a position and/or orientation by the support device in the holding mode; a release device that is communicatively connected with the control device and which is configured to switch the support device from the holding mode into the movement mode; wherein the control device is configured to switch the support device from the movement mode into the holding mode if the supported body part of the operator takes an at least substantially constant position and/or orientation for a predetermined time.

The holding system according to the present disclosure comprises a support device for holding at least a body part of the operator and a release device. Particularly, the support device is configured to hold at least a body part, such as one or both arms, legs, the upper body or the entire body. The support device comprises a control device that is configured to switch the support device between a movement mode and a holding mode, wherein the supported body part of the operator is movable in the movement mode at least in one adjustment direction and is held in the holding mode in a position and/or orientation by the support device. An adjustment direction can be, for example, the seat height of the operator, a lateral inclination angle or an upper body inclination angle of the sitting operator. The release device is communicatively connected with the control device and is configured to switch the support device from the holding mode into the movement mode, particularly if a respective signal is received from the release device. The control device is configured to switch the support device from the movement mode in the holding mode if the supported body part of the operator takes a substantially constant position and/or orientation during a predetermined time. The predetermined time can be in a range of, for example, between 0.5 and 4 s, preferably between 1 and 3 s, preferably between 2 and 3 s or also minimum 5 s. The predetermined time during which the body part of the operator has to rest in a constant position, so that the control device switches from the movement mode into the holding mode can be adjusted to a period that is found to be comfortable by the operator. The supported body part of the operator particularly takes an at least substantially constant position, if the movement velocity of the body part in the adjustment direction is approximately 0 m/s, that is within a tolerance range around the value 0 m/s. The limit values of the tolerance range can be, for example, ±0.2 m/s, ±0.1 m/s or ±0.05 m/s. In addition, the limit values of the tolerance range can be adjusted as well as the time period in a manner that is found comfortable for the operator.

The operator can switch the support device from the holding mode into the movement mode by actuating the release device and in this manner move the supported body part in an adjustment direction. As soon as the operator is content with the position of the supported body part, he can rest in this position. The control device detects that the supported body part of the operator rests in a constant position and switches from the movement mode into the holding mode. This allows a specifically direct and intuitively operation of the support device by means of body movements of the operator. A complicated operating unit for adjusting the support device can be omitted whereby the risk of an incorrect operation of the support device is reduced. In the movement mode the operator can take a desired position and/or orientation that is set after termination of a short time during which the operator rests in this position. The simple adjustment of the operator's position, which is held in the holding mode by means of the support device, makes time-consuming repositionings of the support device unnecessary. The operator can thus concentrate on the operation and is less diverted by the operation of the holding system. In doing so, the operation can be carried out in shorter time with a potentially higher quality.

A particularity of the inventive holding system is the control device that switches the support device from the movement mode into the holding mode if the supported body part of the operator rests in one position for a preset time.

During and/or after the release of the movement mode the control device can additionally produce an acoustical and/or optical signal that indicates the release of the movement mode to the operator. Between releasing the movement mode and the switching of the support device from the holding mode into the movement mode, in addition, a preset delay time can be provided. This is particularly advantageous if the operator is in a position in which he would be unstable in the movement mode. The delay time can be adjusted and can be, for example, minimum 3 s, 2 s, 1 s, 0.5 s or 0.3 s.

Preferably, the support device comprises a sensor unit for detection of movements of the supported body part, wherein the control device is configured to receive sensor data from the sensor unit and to switch the support device from the movement mode into the holding mode based on the sensor data. The sensor unit allows the control device to determine based on the sensor data whether the supported body part of the operator does not move anymore. The sensor unit can detect the movements of the supported body part directly or indirectly, for example, based on movements of individual parts, such as struts, rods or hinges of the support device. The sensor unit can comprise, for example, pressure sensors in a lateral support or at other positions. For example, the sensor unit can also comprise a separate position sensor that is attached to a piston rod of a blockable gas pressure spring that supports the height adjustment of the operating chair. For example, the sensor unit can, however, also comprise a sensor or a circuit arranged in an actuator that monitors the actuator, which is provided for supported movement of parts of the support device, for example, in that motor currents of the actuator and/or a voltage applied to the actuator are detected and evaluated.

It is preferred that the release device comprises at least one operating element for releasing the movement mode. For example, the operating element can comprise an electrical switch, an electrical button or a light barrier or an inductive or capacitive sensor. The operating element of the release device is preferably arranged in the foot range of the operator sitting on the operating chair, so that the operator can release the movement mode by a foot movement. It is preferred that the operating element is equipped with a light barrier.

The release device is preferably arranged so that the operating element can be reached by the operator at least with the foot. In doing so, the operator can switch the support device from the holding mode into the movement mode and from the movement mode into the holding mode without using his hands.

In a specific embodiment, the release device preferably comprises at least two operating elements that are assigned to different adjustment directions of the support device in the movement mode. For example, a first operating element can be arranged so that the operator can reach the latter with his right foot. The first operating element can be configured, for example, to release the adjustment of a height and/or a lateral pivot angle of the operating chair. The second operating element can be arranged, for example, so that the operator can reach the latter with his left foot. The second operating element can be configured, for example, to release the movement mode for the adjustment of an upper body inclination angle of the operator. For example, the operating chair can comprise a base column that can be configured to be exclusively adjustable in height and pivotable in lateral direction. On the base column additional support elements can be attached, such as an upper body retaining device, at least one, preferably multiple lateral supports and/or arm rests and the seat. The additional support elements are preferably attached in an upper end section of the base column.

Particularly, the support device comprises at least one actuator that is configured to transfer the support device at least in one adjustment direction in the holding mode and/or the movement mode. For example, the at least one actuator can be an actuator, which presses on a release pin of a gas pressure spring in the movement mode. The actuator can in addition comprise one or more electrical servo motors, which are configured, for example, to move a part of the support device.

In an embodiment the support device comprises an operating chair having a seat and a first adjustment device for adjusting a seat height. The operator can sit down on the seat during the operation. Preferably, the sensor unit comprises at least one sensor element that is located in the first adjustment device, whereby the sensor element is configured to detect a seat height.

Preferably the first adjustment device is configured to support a movement of the operator in the movement mode by means of a force. The supporting force thereby counteracts the force of gravity of the supported body part of the operator. For example, the first adjustment device can comprise a blockable gas pressure spring or a jack screw driven by an electric motor.

The operating chair particularly comprises a second adjustment device for adjustment of a lateral seat inclination, at least one lateral support and at least one force sensor unit arranged in the lateral support for detection of lateral forces acting onto the lateral support. Preferably, the lateral support extends laterally next to the seat of the operating chair. The force sensor unit is preferably arranged on an inner side of the lateral support facing the seat so that the force sensor unit detects a force with which the upper body of the operator sitting on the seat presses laterally against the lateral support, for example, if the operator moves his upper body laterally. The sensor signal of the force sensor unit can therefore be used to detect a movement of the operator in lateral direction.

Preferably, the second adjustment device is configured to adjust a lateral inclination angle of the operating chair based on sensor signals of the force sensor unit. The operator can in this way adjust a desired lateral inclination angle of the operating chair as simple and intuitively as possible with his upper body in that he displaces his weight, so that his upper body presses against the lateral support. Preferably, the operating chair comprises an additional lateral support, having at least one additional force sensor unit arranged in the additional lateral support for detection of forces that laterally act on the additional lateral support. The additional lateral support extends preferably also laterally next to the seat of the operating chair on an opposite side of the operating chair. The additional force sensor unit is preferably arranged at an inner side of the additional lateral support facing the seat, so that the additional force sensor unit detects a force with which the upper body of the operator sitting on the seat presses laterally against the additional lateral support. Moreover, a force variation, such as a reduction of the force, can be detected in order to initiate erecting of the operating chair. If the operator presses with the upper body against the force sensor unit in the lateral support that is arranged on the right side, the second adjustment device pivots the operating chair to the right, while the second adjustment device pivots the operating chair to the left if the operator presses with his upper body against the additional force sensor unit in the additional lateral support arranged on his left side.

Preferably, the control device is configured to switch the second adjustment device from the movement mode into the holding mode if a sensor signal of the force sensor unit is at least substantially 0 N for the predetermined time. If an additional force sensor unit is located in the additional lateral support, the control unit is configured to switch the second adjustment device from the movement mode into the holding mode if the sensor signal of both force sensor units is at least substantially 0 N for the predetermined time.

In another embodiment, the operating chair comprises an upper body retaining device having a traction means, wherein the traction means is releasably connectable to a back part of a harness that can be worn by the operator. The traction means is preferably a belt, a wire rope with or without coating or the like. The upper body retaining device is preferably arranged, so that the upper body of the operator is retained in an inclination angle in the holding mode. The upper body retaining device is preferably arranged behind the operating chair, for example on the level of the shoulder of the operator sitting on the operating chair. The upper body retaining device is particularly configured to keep the traction means tightly also in the movement mode, so that a slacking traction means is avoided and the risk of injury for the operator and/or for bystanders accompanying therewith can be reduced.

Additionally or alternatively, the control device can be configured to switch the upper body retaining device into a hover mode, wherein the upper body retaining device is in the hover mode configured to apply a traction force on the upper body of the operator via the traction means and the harness. By means of the traction force, movements of the operator are supported in which he inclines his upper body. For example, upon activation of the release device, the control device can be configured to activate the hover mode of the upper body retaining device for a predetermined duration. For example, in this manner the operator can keep the operating element pressed for a predetermined duration, such as for 0.5 s, 1 s or 1.5 s. If the operating element comprises a light barrier, the operator can activate the hover mode accordingly with his foot in that he leaves the foot for a predetermined duration in the release range of the light barrier. The operator can move freely and in accordance with the operation requirements due to the permanently applied resetting force that is perceived by the operator as slight resistance, without being required to repeatedly actuate an operating unit for a position and/or orientation change. The operator can in this manner directly move in the hover mode without having to put down the instruments, whereby he is nevertheless relieved or supported to a certain degree. The resetting force can be 60%, 70%, 80%, or 90% of the gravity force of the supported body part, for example.

Preferably the control device is configured to switch the support device in case of a repeated actuation of the release device from the hover mode into the holding mode. The operator can in this way deactivate the hover mode by a repeated actuation of the release device.

For example, the upper body retaining device can comprise an electrically driven winch on or from which the traction means is wound. The electrical drive of the winch for the traction means can be particularly controlled, so that it drives the winch with an approximately constant torque in wind-up direction, so that an approximately constant traction force is applied on the traction means. The electrically driven winch preferably comprises a gear having a transmission ratio, whereby the winch is configured, for example, in self-locking manner.

The object is also solved by means of the method for holding an operator according to claim 14:

The method according to the present disclosure comprises the following steps: holding at least one supported body part of the operator in a position and/or orientation; releasing a movement mode in which the supported body part of the operator can be moved at least in an adjustment direction; and switching from the movement mode into the holding mode if the supported body part of the operator takes an at least substantially constant position and/or orientation for a predetermined time.

Preferably, in the holding mode the release of the movement mode can be carried out by foot.

All of the features and advantages that have been described in relation to the holding system according to the present disclosure can be similarly applied to the method according to the present disclosure.

### BRIEF DESCRIPTION OF THE DRAWINGS

Additional details of advantageous embodiments or details of the present disclosure are derived from the drawing, the description and the dependent claims. The drawing shows:

FIG. 1 a schematic three-dimensional view of an embodiment of a holding system according to the present disclosure,

FIG. 2 a schematic block diagram of an embodiment of the holding system,

FIG. 3 a schematic block diagram of another embodiment of the holding system,

FIG. 4 a schematic block diagram of another embodiment of the holding system,

FIG. 5 a flow diagram for an embodiment of the method according to the present disclosure, and

FIG. 6 an example of sensor data during adjustment of the holding system in the movement mode.

### DETAILED DESCRIPTION

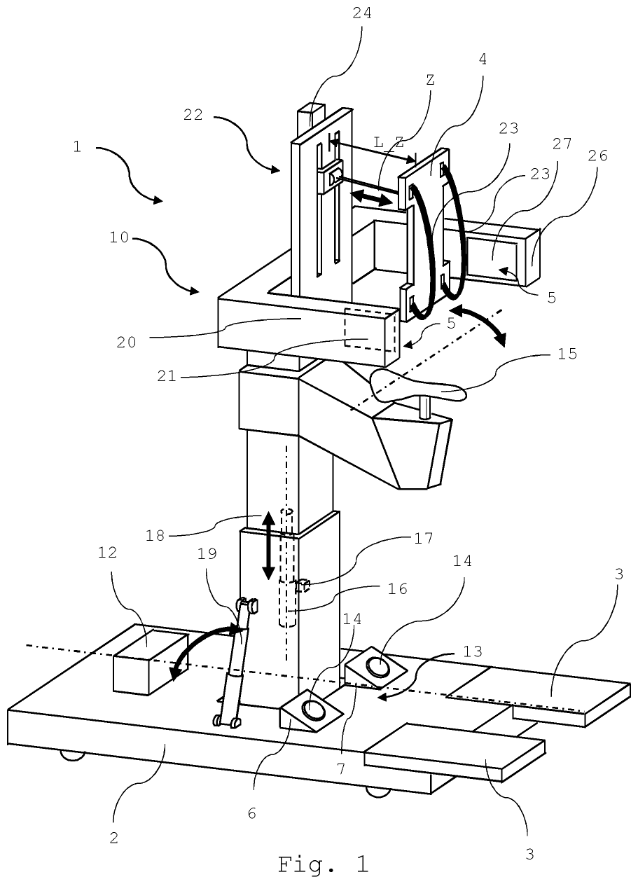

In FIG. 1, an embodiment of the holding system 1 is illustrated that serves to hold an operator in a sitting position, wherein the upper body of the operator is held by means of a harness 23 having a back part 4, which can be worn by the operator.

The holding system 1 comprises a support device 10, having an operating chair 18, which is adjustable in height and laterally pivotable. The operating chair comprises a seat 15, and an upper body retaining device 22, having a traction means Z. The operator can sit down on seat 15. Before the operator sits down on operating chair 18, he can put on a harness 23 having a back part 4, which can be releasably coupled with traction means Z of the upper body retaining device 22. So, the operator can lean forward, whereby his upper body is retained by traction means Z. The adjusted length L_Z of traction means Z, between the upper body retaining device 22 and the back part 4 of harness 23 thereby determines the inclination angle of the upper body of the operator.

The support device 10 depicted in FIG. 1 is arranged on a drivable platform 2 that in addition comprises two foot rests 3, on which the operator can place his feet during the operation. The foot rests 3 can in addition serve as holder for foot pedals for operation of instruments, such as surgical instruments.

The operating chair 18 is equipped with a first adjustment device 16 and a second adjustment device 19.

In the present example, the first adjustment device 16 comprises a blockable gas pressure spring. Moreover, in the first adjustment device 16, a sensor element 17 is located that detects the seat height, that is the height of seat 15 above platform 2.

The second adjustment device 19 comprises, in this example, two linear drives (only the one in front is depicted), which are respectively arranged laterally next to the operating chair 18. The two linear drives are attached to the operating chair 18 and the platform 2, so that a length change of the linear drives result in a pivot movement of the operating chair. The linear drives are operated to move in opposite direction, so that in case of a positive length change of one linear drive, the other linear drive carries out a negative length change.

Above seat 15 the support device 10 comprises a lateral support 20 and an additional lateral support 26 that extend laterally next to the seat. In the lateral support 20, a force sensor unit 29 is located that is arranged on an inner side 5 of lateral support 20 facing the seat, so that the force sensor unit 21 detects a force with which the upper body of the operator sitting on seat 15 presses laterally against the lateral support 20. Similarly, an additional force sensor unit 27 is located in the additional lateral support 26.

Between the two lateral supports 20, 26 behind seat 15, an upper body retaining system 22 extends in addition in vertical direction compressing a mast 24. The upper body retaining device 22 comprises a traction means Z that can be attached to a back part 4 of a harness 23 that can be worn on the upper body by the operator. The traction means Z of the upper body retaining device 22 can be wound up or wound down, so that via the length L_Z of traction means Z, an inclination angle of the upper body of the operator can be adjusted. Additionally, the upper body retaining device 22 comprises adjustment means with which the position can be defined at which the traction means Z exits from the upper body retaining device 22. The adjustment means can adjust and/or lock the position, for example, in a stepless manner or in discrete steps. In doing so, the upper body retaining device 22 can be adapted to the body height of the operator. It is preferred that the upper body retaining device 22 can be adjusted, so that the position at which traction means Z exits from the upper body retaining device 22 behind the operator sitting on the seat is arranged minimum on the level of the shoulders of the operator or higher.

The support device 10 additionally comprises a control device 12 that is configured to switch individual actuators of support device 10 between a movement mode B and a holding mode H. In the movement mode B, the supported body part of the operator can be moved in an adjustment direction.

The support device 10 comprises in addition a release device 13 that is communicatively connected with the control device 12. The release device 13 is arranged on an upper side of platform 2. The release device 13 comprises in this embodiment two operating elements 14, namely a first foot operating element 6 and a second foot operating element 7. The foot operating elements 6 and 7 comprise on their upper side a release area in which a foot of the operator is detected. The operating elements 14 and the release device 13 are arranged below the seat 15 between the foot rests 3 and the operating chair 18.

The first foot operating element 6 can be reached with a right foot of the operator and the second foot operating element 7 can be reached with a left foot of the operator. The release device 13 can also comprise one or more light barriers defining a range in which a foot of the operator can be detected.

The control device 12 is configured in case of an actuation of the release device 13 to switch the support device 10 from the holding mode H into the movement mode B. After actuating the release device 13 once again, the control device 12 switches the support device 10 back again from the movement mode B into the holding mode H.

In the present example, the first foot operating element 6 for releasing movement mode B for the first adjustment device 16 and the second adjustment device 19 is assigned to the first foot operating element 6. The second foot operating element 7 is, however, assigned to releasing movement mode B of upper body retaining device 22.

The release device 13 can comprise additional foot operating elements, so that, for example, the release of movement mode B for the first adjustment device 16 and the second adjustment device 19 can be assigned to separate foot operating elements.

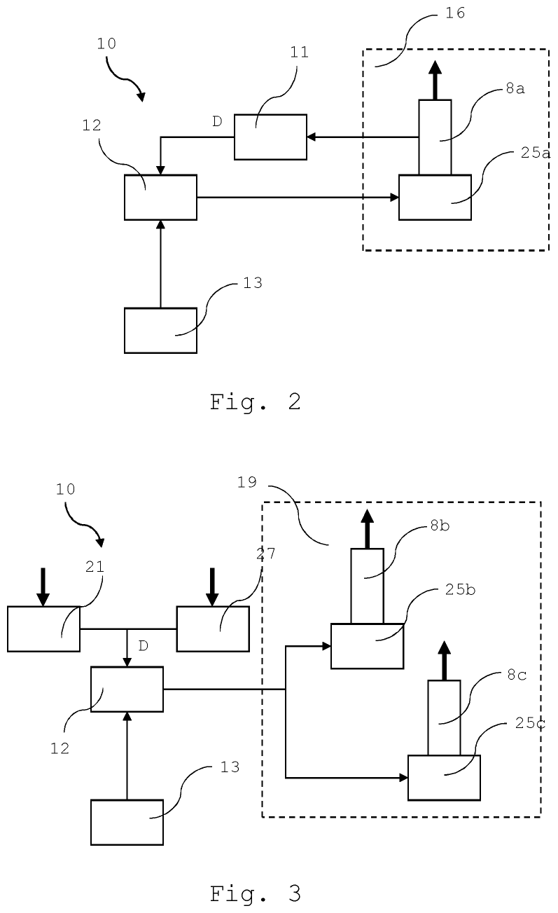

A block diagram of an embodiment of holding system 1 is illustrated in FIG. 2. The support device 10 comprises an operating chair 18 having a seat 15 as well as a first adjustment device 16 having minimum one actuator 25a. The actuator 25a comprises a drive 8a that is configured to create a movement and/or a force of the first adjustment device 16.

In this example, support device 10 comprises in addition a sensor unit 17, which is configured to detect a measurement parameter of drive 8a. Such a measurement parameter can be, for example, a position, an angular position, a velocity, an angular velocity, an acceleration, an angular acceleration, a force or a torque of drive 8a.

The sensor unit 17 is communicatively connected with control device 12, so that the control device 12 is configured to receive and evaluate sensor data D of sensor unit 17. The control device 12 is in addition connected with release device 13 with minimum one operating element 14. The control device 12 is configured to receive a release signal from release device 13 in case of actuation of the operating element 14. If the control device 12 receives the release signal from release device 13, it switches the mode in which the actuator 25a operates. If the actuator 25a has been operated up to present in the holding mode H, the control device 12 thus switches in the movement mode B. In movement mode B the actuator 25a is controlled, so that first adjustment device 16 can be at least moved in an adjustment direction by the operator. If the first adjustment device 16 comprises, for example, a gas pressure spring and is configured to adjust the height of the operating chair, the actuator 25a can be configured, for example, to press on a release pin of the gas pressure spring in order to transfer the gas pressure spring into the movement mode B. If it is determined by control device 12 that the height of the operating chair 18 remains at least substantially constant for a predetermined time I, it is switched from movement mode B into holding mode H.

FIG. 3 illustrates another embodiment of holding system 1. For the embodiment depicted in FIG. 3, the above description applies based on the already explained reference numerals accordingly. It applies in addition: In this embodiment operating chair 18 comprises a second adjustment device 19 comprising two actuators 25b, 25c with respectively one drive 8b, 8c. The control device 12 is connected to the force sensor unit 21 located in the lateral support 20 and the additional force sensor unit 27 located in the additional lateral support 26. If the second adjustment device 19 is operated in the holding mode H, actuators 25b, 25c remain in their actual position. If control device 12 now receives a release signal from release device 13, control device 12 is configured to switch the second adjustment device 19 into the movement mode B. The actuators 25b, 25c are controlled based on sensor data D of force sensor units 21, 27. If both force sensor units 21, 27 transmit a measurement value of at least substantially 0 N to control device 12 for a predetermined time I, control device 12 switches from movement mode B into holding mode H.

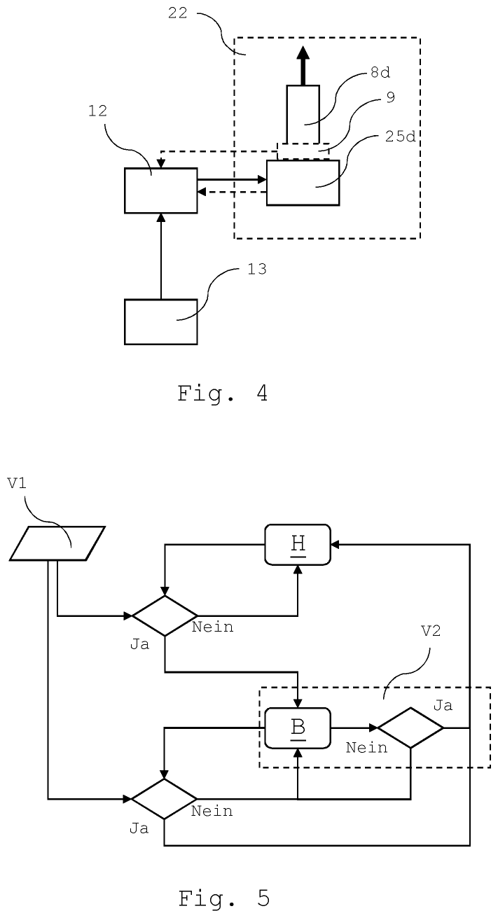

Another embodiment is illustrated in FIG. 4. Also for this embodiment, the above description applies accordingly. It applies in addition: The support device 10 with the operating chair 18 comprises an upper body retaining device 22 in this example. The control device 12 can be configured in this embodiment to directly conclude on the position of the actuator due to the motor current of the actuator and/or a voltage applied to the actuator 25d. Additionally or alternatively, the actuator 25d can comprise a stepper motor or a servo motor having an angle sensor 9. Both alternatives are shown in FIG. 4 in dashed lines.

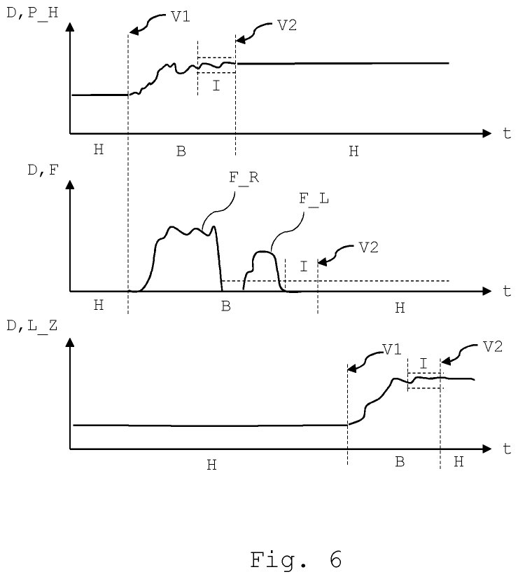

FIG. 5 shows a flow diagram for the inventive method for holding an operator. The support device 10 of holding system 1 is first in the holding mode H. In this step, the supported body part of the operator is held in a position and/or orientation. In step V1 now the movement mode B is released, in which the supported body part of the operator can be moved in an adjustment direction. If the movement mode B is not released, holding system one remains further in the holding mode H. If the support device 10 of holding system 1 is in the movement mode B and a condition change is released by the operator (method step V1), then the support device 10 is switched from the movement mode B into the holding mode H.

If for a predetermined time I the supported body part of the operator takes at least substantially a constant position and/or orientation, the support device 2 is switched back by control device 12 from the movement mode B into the holding mode H in the second method step V2.

The holding system 1 for an operator described so far operates as follows:

For carrying out an operation holding system 1 is driven to the operating table by means of a not further illustrated operating unit, whereby the operating unit can comprise, for example, at least one control lever as well as additional control buttons or other control elements. The operator puts on harness 23 with back part 4, enters platform 2 and sits down on seat 15 of operating chair 18. Before or after sitting down back part 4 and traction means Z are mechanically releasably coupled with each other.

On the back part 4 a first coupling element is attached, which is complementary to a second coupling element attached to an end of traction means Z. The first coupling element and the second coupling element comprise preferably magnet elements so that the first and the second coupling element move toward one another in a pretensioning position during coupling together. In the pretensioning position both coupling elements can be manually pressed into a latched position in which the two coupling elements releasably connect traction means Z with back part 26 of harness 25. If the operator first puts on harness 25 and thereafter puts on a preferably sterile surgical gown having a back opening, an additional person can connect first coupling element with second coupling element under the surgical gown also without the need to see both coupling elements. This accelerates the processes before the operation.

The support device 10, that is the operating chair 18 adjustable in height and laterally pivotable and the upper body retaining device 22, is first in the holding mode H.

In FIG. 6 an example for sensor data D over time t is illustrated, wherein in the top line sensor data D of sensor unit 17 for the seat height, in the middle line sensor data D of force sensor units 21, 27 and in the bottom line the length L_Z of traction means between back part 4 and the upper body retaining device 22 are exemplarily illustrated.

The first adjustment device 16 is first in holding mode H. Now release device 13 for first adjustment device 16 is released by the operator by means of actuating the operating element 14 with the foot (method step V1). The first adjustment device 16 is switched into movement mode B, so that the operator can adjust the height P_H of operating chair 18 in that he loads or relieves the seat 15 of operating chair 18 with his legs. During the adjustment of the height of seat 15 the height P_H is detected by sensor unit 17 and transmitted to control device 12. As soon as the detected height P_H remains at least substantially constant for a predetermined time I, control device 12 switches the first adjustment device 16 from movement mode B into holding mode H (method step V2).

In the second line of FIG. 6 the process described before with relation to the first adjustment device is now described in relation to the second adjustment device 19. The sensor data D used for control of the second adjustment device 19 are the force sensor units 21, 27 in the lateral supports 20, 26.

The second adjustment device 19 is also in the holding mode H. By releasing the release device 13 in the method step V1, the second adjustment device 19 is switched from holding mode H into movement mode B by control device 12. In this example the operator presses first with his upper body against the force sensor unit 21 in the right lateral support 20. The force acting on the lateral support 20 is detected by force sensor unit 21. The force sensor unit 21 transmits a respective force signal F_R to control device 12. Then operator presses with his upper body against force sensor unit 27 located in the left lateral support 26, so that it forwards a force signal F_L to control device 12. The control device 12 is configured to evaluate the force signals F_R and F_L and to switch from movement mode B into holding mode H, as soon as the force signals F_R, F_L have at least substantially the value 0 N for a predetermined time I (method step V2).

In the lowest line of FIG. 6 the above-described procedure is now described with relation to the upper body retaining device 22. Also here, starting with holding mode H in method step V1, the movement mode B for the upper body retaining device 22 is released. The operator is now able to change the inclination of his upper body whereby the length L_Z of traction means Z changes accordingly. In the upper body retaining device 22 a respective sensor unit 22 is located configured to detect the length L_Z of traction means Z. The sensor unit 11 sends a respective sensor signal to control device 12 that is configured to evaluate the sensor signal. As soon as the control device 12 detects that the change of the length L_Z of traction means Z does at least substantially no longer change, it switches the upper body retaining device 22 from movement mode B into holding mode H (method step V2).

The holding system 1 according to the present disclosure with support device 10 for holding at least one body part of the operator and a release device 13 serves to hold at least a body part of the operator in an adjustable position and/or orientation. The support device 10 comprises a control device 12 configured to switch the support device 10 between a movement mode B and a holding mode H, wherein the supported body part of the operator can be moved at least in one adjustment direction in the movement mode B and is held in an actual position by the support device in the holding mode H. The control device 12 is configured to switch the support device 10 from the movement mode B into the holding mode H, if for a predetermined time the supported body part of the operator remains in an at least substantially constant position. This allows a particularly direct and intuitive operation of the support device by means of body movements of the operator. The operator can take a position and/or orientation in the movement mode that is set after termination of a short time during which the operator remains in this position.

### REFERENCE SIGNS

1 holding system
2 platform
3 foot rests
4 back part
5 inner side of the lateral support facing the seat
6 first foot operating element
7 second foot operating element
8a . . . , 8d drives of actuators
9 position sensor
10 support arrangement
11 sensor unit
12 control device
13 release device
14 operating element
15 seat
16 first adjustment device
17 additional inner side of the additional lateral support facing the seat
18 operating chair
19 second adjustment device
20 lateral support
21 force sensor unit
22 upper body retaining device
23 harness
24 mast
25a . . . , 25d actuators
26 additional lateral support
27 additional force sensor unit
B movement mode
D sensor data
F_R force signal of force sensor unit in the right lateral support
F_L force signal of force sensor unit in the left lateral support
H holding mode
I predetermined time
L_Z length of traction means
P actual position
P_H height of seat
S hover mode
t time
V1 holding at least one supported body part of surgeon in the holding mode
V2 start of movement mode
V3 switching from movement mode into holding mode

1. A holding system for an operator comprising:
a support device for holding at least one body part of the operator, which comprises a control device that is configured to switch the support device between a movement mode and a holding mode, wherein the supported body part of the operator can be moved in an adjustment direction in the movement mode and is held in a position and/or orientation by the support device in the holding mode;
a release device that is communicatively connected with the control device and which is configured to switch the support device from the holding mode into the movement mode;
wherein the control device is configured to switch the support device from the movement mode into the holding mode if the supported body part of the operator takes an at least substantially constant position and/or orientation for a predetermined time.

2. The holding system according to claim 1, wherein the support device comprises a sensor unit for detection of movements of the supported body part, wherein the control device is configured to receive sensor data from the sensor unit and to switch the support device from the movement mode into the holding mode based on the sensor data.

3. The holding system according to claim 1, wherein the release device comprises at least one operating element for releasing the movement mode.

4. The holding system according to claim 1, wherein the release device is arranged, so that at least one operating element can be reached by the operator at least with a foot.

5. The holding system according to claim 1, wherein the support device comprises at least one actuator that is configured to transfer the support device at least in one adjustment direction in the holding mode and/or in the movement mode.

6. The holding system according to claim 2, wherein the support device comprises an operating chair having a seat and a first adjustment device for adjusting a seat height.

7. The holding system according to claim 6, wherein the sensor unit comprises at least one sensor element located in the first adjustment device, wherein the sensor element is configured to detect a seat height.

8. The holding system according to claim 6, wherein the first adjustment device is configured to support a movement of the operator by means of a force in the movement mode.

9. The holding system according to claim 6, wherein the operating chair comprises a second adjustment device for adjusting a lateral seat inclination, at least one lateral support and at least one force sensor unit arranged in the lateral support for detection of a force acting laterally onto the lateral support.

10. The holding system according to claim 9, wherein the second adjustment device is configured to adjust a lateral inclination angle of the operating chair in the movement mode based on sensor signals of the force sensor unit,
wherein the control device is configured to switch the second adjustment device from the movement mode into the holding mode if a sensor signal of the force sensor unit is at least substantially 0 N for the predetermined time.

11. The holding system according to claim 6, wherein the operating chair comprises an upper body retaining device having a traction means, wherein the traction means can be releasably attached to a back part of a harness that can be worn by the operator,
wherein the upper body retaining device is arranged, so that an upper body of the operator is retained in the holding mode in an inclination angle.

12. The holding system according to claim 11, wherein the control device is configured for a predetermined time duration to switch the upper body retaining device into a hover mode in case of a release of the release device, wherein in the hover mode the upper body retaining device is configured to apply a traction force onto the upper body of the operator via the traction means and the harness.

13. The holding system according to claim 12, wherein the control device is configured to switch from the hover mode into the holding mode in case the release device is released once again.

14. A method for holding an operator, by means of a holding system for an operator comprising: a support device for holding at least one body part of the operator, which comprises a control device that is configured to switch the support device between a movement mode and a holding mode, wherein the supported body part of the operator can be moved in an adjustment direction in the movement mode and is held in a position and/or orientation by the support device in the holding mode; a release device that is communicatively connected with the control device and which is configured to switch the support device from the holding mode into the movement mode; wherein the control device is configured to switch the support device from the movement mode into the holding mode if the supported body part of the operator takes an at least substantially constant position and/or orientation for a predetermined time, wherein the method comprises the following steps:
holding the at least one supported body part of the operator in the position and/or orientation;
releasing the movement mode in which the supported body part of the operator can be moved at least in one adjustment direction; as well as
switching from the movement mode into the holding mode, if the supported body part of the operator takes the at least substantially constant position and/or orientation for the predetermined time.

15. The method according to claim 14, wherein releasing of the movement mode in the holding mode can be carried out by foot.

16. The holding system according to claim 2, wherein the release device comprises at least one operating element for releasing the movement mode.

17. The holding system according to claim 16, wherein the release device is arranged, so that at least one operating element can be reached by the operator at least with a foot.

18. The holding system according to claim 17, wherein the support device comprises at least one actuator that is configured to transfer the support device at least in one adjustment direction in the holding mode and/or in the movement mode.

19. The holding system according to claim 18, wherein the support device comprises an operating chair having a seat and a first adjustment device for adjusting a seat height.

20. The holding system according to claim 19, wherein the sensor unit comprises at least one sensor element located in the first adjustment device, wherein the sensor element is configured to detect a seat height.
